(19) United States
(12) Patent Application Publication
(10) Pub. No.: US 2021/0321900 A1
(43) Pub. Date: Oct. 21, 2021

(54) Methods and Systems for Determining Body Composition of Biological Bodies Using a Resonant Cavity

(71) Applicant: Lisa Michelle Kotowski, Rancho Palos Verdes, CA (US)

(72) Inventor: Lisa Michelle Kotowski, Rancho Palos Verdes, CA (US)

(21) Appl. No.: 17/229,515

(22) Filed: Apr. 13, 2021

Related U.S. Application Data
(60) Provisional application No. 63/010,265, filed on Apr. 15, 2020.

(51) Int. Cl. A61B 5/0537; A61B 5/00; G16H 50/30; G01R 29/08

(52) U.S. Cl. CPC A61B 5/0537; A61B 5/7275; G01R 29/0814; G01R 29/0878; G16H 50/30

(57) ABSTRACT

Systems and methods for generating a body composition measurement of a biological body include a resonant cavity, antennae used to measure resonance of the empty cavity and a biological body in the cavity, a network analyzer, and a controller configured to generate a body composition measurement based on the resonance measurements. The body composition measurement is used to determine risk assessments of developing metabolic disease, particularly for children, based on body fat composition. Height, weight, and body shape are also measured by the systems and methods to contribute to the risk assessment.

### CROSS-REFERENCE

The present application relies on U.S. Patent Provisional Application No. 63/010,265, titled “Methods and Systems for Determining Body Composition of Biological Bodies Using a Resonant Cavity” and filed on Apr. 15, 2020, for priority. The above-mentioned application is herein incorporated by reference in its entirety.

### FIELD

The present specification is directed toward methods and systems for using resonant cavities to accurately determine the composition, including fat mass and fat free mass percentages, of a biological body. The present application is also directed toward an integrated body measurement system for humans to accurately, and concurrently, determine weight, height, and body composition, including fat and muscle percentages.

### BACKGROUND

Early growth from birth to two years of age has been found to have a strong effect on the risk of developing metabolic diseases, including but not limited to diabetes, non-alcoholic fatty liver disease, and obesity, later in life. Body composition is an underutilized measurement of individual health and should be used as an annual measure to track individual growth quality and track personal health goals. Growth assessment during early childhood, particularly birth through 5 years of age, is largely based on height, weight, and anthropometric equations with insufficient attention to relative partitioning of weight into lean mass or fat mass. At birth, length, weight, and head circumference might be measured as indicators of the outcome of pregnancy, while measurements of weight and length during infancy and throughout childhood are used to assess growth by the World Health Organization (WHO) Child Growth and Center for Disease Control and Prevention (CDC) standards. The National Institute of Health (NIH) recommends the WHO growth chart for children ages 0-2 and the CDC growth chart children ages 2 and older. Infants of similar height and weight at birth can vary substantially in body composition. For example, a thin-fat phenotype has been described in Indian children. These infants tend to be thin and short but have more body fat proportionally distributed throughout their bodies as compared to their European counterparts. The difference in body composition appears to be related to birth size and indicates an innate metabolic difference and a greater risk for diabetes and other metabolic diseases later in life. Observations like these emphasize the importance of detailed characterizations of body structure and composition from infancy to identify higher risk of metabolic disease later in life.

Body weight and height are quick, simple, and informative measurements which provide short-term insight into nutritional status and metabolic disease risk. However, body weight and height alone do not properly reflect the risks associated with poor quality growth in the long-term assessment of children's health and development. Although good measurements of height and weight provide useful information, it is clear in adults that there is more information about health in the distribution and partitioning of lean mass and fat mass in the body. Definitions of healthy growth need to be extended to include the quality of growth based on body composition measurements in infants and young children.

Several studies have found that infant body composition is an important marker of immediate and lifelong health status. Body composition assessment in early life and throughout growth is urgently needed to understand the associations between quality of growth during early life and later life health status. Growth charts of body composition can be extended into adulthood to evaluate changes in fat and lean mass throughout a participant's life course. This would allow for more effective intervention recommendations and allow for children to grow healthier and happier.

Current technologies fail to adequately measure body composition periodically for individuals between birth and adulthood utilizing non-invasive methods. In early studies, surrogate measures such as body mass index (BMI) and anthropometric equations have been correlated with mortality rates. However, BMI is less indicative of overall health in studies which investigate both adiposity and BMI. Waist circumference or the ratio of waist circumference to hip width is more closely associated with adiposity and adverse outcomes. Total body composition measurements can be estimated from optical body shape, but these methods have not been demonstrated for infants and children. The fundamental measures of lean and fat masses are strongly associated with adverse outcomes and can be derived from total body composition as an indicator of overall health throughout childhood, adolescence, and into adulthood.

Since infant body composition is indicative of lifelong metabolic health, being able to accurately measure body composition is imperative for assessing infant health status. However, reliable measurements of body composition in infancy and early life represent a technically challenging area. Current methods of measuring body composition in infancy and early childhood include dual-energy X-ray absorptiometry (DXA), air displacement plethysmography (ADP), bio-impedance analysis (BIA), and total body water (TBW) deuterium dilution. The ideal body composition measurement will not utilize ionizing radiation, not be sensitive to motion artifacts, be inexpensive, be able to periodically measure composition throughout a child's life, and be sensitive to composition. Of the four approaches presented above, none have all the desirable characteristics with minimal inconveniences.

DXA is the current gold standard for body composition measurements from infancy through adulthood. However, DXA utilizes ionizing radiation and, as such, it is not recommended to take frequent measurements. ADP doesn't utilize ionizing radiation and isn't sensitive to motion artifacts, but the required device is expensive. Additionally, there is a significant age gap for device utilization from 2-5 years when a body size is between the PEA POD® ADP device system maximum capacity and the BOD POD® ADP device system minimum capacity, wherein the device cannot be used to monitor changes in body composition. BIA provides a technique which can be measured frequently but typically provides the least accurate result. TBW measurements through deuterium dilution provide reproducible and accurate results but is expensive to conduct. The deuterium dilution method requires biological sample collections which must be processed in a laboratory through a labor intensive process, which makes deuterium dilution unreasonable to measure frequently. Further, these current methods for measuring body composition are often noisy or aesthetically frightening. As the current technologies used for measuring infant body composition each have their merits and drawbacks, a new method for measuring body composition from birth through adulthood is needed.

Hence, there is need for a device and method to periodically measure body composition continuously from birth through adulthood. Further, a comprehensive pediatric growth evaluation system that integrates height, weight, and body composition measurements, which does not use ionizing radiation, is non-invasive, is not sensitive to motion artifacts, is sensitive to composition, is inexpensive, and is able to periodically measure composition throughout an individual's entire life, is needed.

### SUMMARY

The present specification discloses resonant cavity perturbation (RCP) devices configured to measure total body water, and optionally, concurrently measure body shape, height, and weight, using a single measurement procedure. The devices comprise a rectangular resonant cavity which operates in the radio frequency regime. Antennae are mounted to the walls and ceiling of the device, and are capable of measuring the TEₘₙₚ modes of the resonant cavity. The RCP devices comprise a built-in network analyzer which measures the S parameters of the cavity and software which is able to interpret and translate the radiofrequency data into body composition data.

The cavity is constructed from a conductive material. One side of the cavity is a door comprising a clear conductive material, such as conductive glass or metallic mesh. A plastic, cloth, or non-conductive veneer may be placed throughout the interior and onto the exterior of the cavity for aesthetic purposes. An optional 3D motion tracking technology is mounted to the inside of the cavity, which can accurately measure the shape and height of an individual inside the cavity. The floor of the device may comprise a scale, which has a flexible conductive material around the edges.

The present specification discloses a system for generating a body composition measurement of a biological body, comprising: a resonant cavity comprising a frame and metal material lining the frame, wherein the resonant cavity has a top panel, bottom panel, and four side panels to thereby define an enclosed volume; at least one antenna in the resonant cavity; a network analyzer in data communication with the at least one antenna; and a controller configured to receive data from the network analyzer and generate an output, wherein the output comprises data indicative of the body composition of the biological body.

Optionally, the system further comprises a scale in physical communication with the resonant cavity and configured to measure a weight of the biological body in the enclosed volume, wherein the controller is further configured to receive data from the scale and generate an output, wherein the output comprises data indicative of the weight of the biological body.

Optionally, the system further comprises a height measurement system coupled to the resonant cavity and configured to measure a height of the biological body in the enclosed volume, wherein the controller is further configured to receive data from the height measurement system and generate an output, wherein the output comprises data indicative of the height of the biological body.

Optionally, the system further comprises an optical shape measurement system coupled to the resonant cavity and configured to measure a shape of the biological body in the enclosed volume, wherein the controller is further configured to receive data from the optical shape measurement system and generate an output, wherein the output comprises data indicative of the shape of the biological body.

Optionally, the system further comprises an oscillator in data communication with the network analyzer.

Optionally, at least one of the four side panels is a door and is configured to open and close in order to provide access to the enclosed volume.

Optionally, the system further comprises a second antenna, wherein the at least one antenna and the second antenna are turned to two different orthogonal directions in the resonant cavity. Optionally, the system further comprises a third antenna, wherein the at least one antenna, the second antenna, and the third antenna are each turned to different orthogonal directions in the resonant cavity.

Optionally, the controller is configured to generate the body composition measurement of biological body by measuring a cavity resonance and a cavity Q factor of the resonant cavity when empty, measuring a mass of the biological body, measuring a body resonance and a body Q factor of the biological body, calculating differences or shifts of the cavity resonance relative to the body resonance and the cavity Q factor relative to the body Q factor, adjusting for fill factor of the cavity, calculating a total body water, and estimating a fat mass of the body. Optionally, the controller is further configured to determine a risk assessment of developing metabolic disease based on the generated body composition measurement.

The present specification also discloses a method for generating a body composition measurement of a biological body, comprising: providing a body composition measurement system comprising: a resonant cavity comprising a frame and metal material lining the frame, wherein the resonant cavity has a top panel, bottom panel, and four side panels to thereby define an enclosed volume; at least one antenna in the resonant cavity; a network analyzer in data communication with the at least one antenna; and a controller configured to receive data from the network analyzer and generate an output; measuring a cavity resonance and a cavity Q factor of the cavity when empty; measuring a mass of the biological body placed within the cavity; measuring a body resonance and a body Q factor of the biological body placed within the cavity; determining a differential between the measured resonances and Q factors; measuring a total water content of the biological body by using the determined differential; and generating data indicative of the body composition of the biological body by using the measure mass and total water content of the biological body.

Optionally, the system further comprises a scale in physical communication with the resonant cavity and configured to measure a weight of the biological body in the enclosed volume, and the method further comprises receiving data from the scale and generating an output, wherein the output comprises data indicative of the weight of the biological body.

Optionally, the system further comprises a height measurement system coupled to the resonant cavity and configured to measure a height of the biological body in the enclosed volume, and the method further comprises receiving data from the height measurement system and generating an output, wherein the output comprises data indicative of the height of the biological body.

Optionally, the system further comprises an optical shape measurement system coupled to the resonant cavity and configured to measure a shape of the biological body in the enclosed volume, and the method further comprises receiving data from the optical shape measurement system and generating an output, wherein the output comprises data indicative of the shape of the biological body.

Optionally, the system further comprises an oscillator in data communication with the network analyzer.

Optionally, at least one of the four side panels is a door and is configured to open and close in order to provide access to the enclosed volume.

Optionally, the system further comprises a second antenna, wherein the at least one antenna and the second antenna are turned to two different orthogonal directions in the resonant cavity. Optionally, the system further comprises a third antenna, wherein the at least one antenna, the second antenna, and the third antenna are each turned to different orthogonal directions in the resonant cavity.

Optionally, the controller is configured to generate the body composition measurement of biological body by calculating differences or shifts of the cavity resonance relative to the body resonance and the cavity Q factor relative to the body Q factor, adjusting for fill factor of the cavity, calculating a total body water, and estimating a fat mass of the body.

Optionally, the network analyzer is further configured to determine a risk assessment of developing metabolic disease based on the generated body composition measurement.

The aforementioned and other embodiments of the present shall be described in greater depth in the drawings and detailed description provided below.

### BRIEF DESCRIPTION OF THE DRAWINGS

FIG. 1A is an illustration of an integrated resonant cavity perturbation (RCP) measurement system in accordance with one embodiment of the present specification;

FIG. 1B is an illustration of an integrated resonant cavity perturbation (RCP) measurement system in accordance with another embodiment of the present specification;

FIG. 2 is an illustration of a resonant cavity, network analyzer, and oscillator of an integrated resonant cavity perturbation (RCP) measurement system, in accordance with an embodiment of the present specification;

FIG. 3 is a schematic flow diagram showing the scattering parameters of a network analyzer coupled with a resonant cavity, in accordance with an embodiment of the present specification;

FIG. 4A is a graph showing a response of the resonant characteristics of the resonant cavity, in accordance with an embodiment of the present specification;

FIG. 4B is a graph illustrating a Gaussian-like distribution for the S21 parameter of the resonant cavity in TE₁₁₀ mode, in accordance with an embodiment of the present specification;

FIG. 4C is a graph showing a peak-fit and data for the TE₁₁₀ mode of the resonant cavity obtained by using Gnuplot, in accordance with an embodiment of the present specification;

FIG. 5 is an illustration of an exemplary resonant cavity, in accordance with an embodiment of the present specification;

FIG. 6A is a flowchart illustrating a method for generating a body composition measurement of a biological body, by using a resonant cavity, in accordance with an embodiment of the present specification; and

FIG. 6B is a flowchart illustrating the steps in a method of using an integrated resonant cavity perturbation (RCP) measurement system to measure body composition and provide a risk assessment of developing metabolic disease, in accordance with an embodiment of the present specification.

### DETAILED DESCRIPTION

The present specification discloses the use of a resonant cavity, integrated with shape, height, and weight measurement systems, to provide a holistic view of the weight, height, and composition of a biological body, including humans and animals such as, but not limited to, cattle. The resonant cavity perturbation (RCP) methods disclosed herein do not suffer from the shortcomings of the current methods for measuring body composition. Specifically, RCP provides a cost-effective method with an excellent re-test ability relative to current methods for measuring body composition. The RCP technique is extremely sensitive to water content and is an accurate method for measuring total body water as compared with DXA, BIA, and ADP. The RCP technique provides an inexpensive measurement which can be repeated frequently and, as an advantage, allows infants to be placed in a comfortable space without restraint. In some embodiments, the RCP cavities of the present specification include a non-conductive veneer to create a comforting environment into which infants, toddlers, and children will be more likely to actively enter and remain.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

“Treat,” “treatment,” and variations thereof refer to any reduction in the extent, frequency, or severity of one or more symptoms or signs associated with a condition.

“Duration” and variations thereof refer to the time course of a prescribed measurement, from initiation to conclusion. Over the duration of treatment, a plurality of measurement periods may be prescribed during which one or more prescribed stimuli are administered to, or captured from, the subject.

The term “and/or” means one or all of the listed elements or a combination of any two or more of the listed elements.

In the description and claims of the application, each of the words “comprise” “include” and “have”, and forms thereof, are not necessarily limited to members in a list with which the words may be associated. The terms “comprises” and variations thereof do not have a limiting meaning where these terms appear in the description and claims.

Unless otherwise specified, “a,” “an,” “the,” “one or more,” and “at least one” are used interchangeably and mean one or more than one.

The term “controller” refers to an integrated hardware and software system defined by a plurality of processing elements, such as integrated circuits, application specific integrated circuits, and/or field programmable gate arrays, in data communication with memory elements, such as random access memory or read only memory where one or more processing elements are configured to execute programmatic instructions stored in one or more memory elements.

“S-parameters” describe the input-output relationship between ports (or terminals) in an electrical system. If a system has two ports (i.e. Port 1 and Port 2), then S12 represents the power transferred from Port 2 to Port 1. S21 represents the power transferred from Port 1 to Port 2. In general, SNM represents the power transferred from Port M to Port N in a multi-port network.

In embodiments, a “port” is defined as any place where we can deliver voltage and current. S11 represents how much power is reflected from the antenna, and hence is known as the reflection coefficient. Note that in general S-parameters are a function of frequency (i.e. vary with frequency).

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

It should be appreciated that the devices and embodiments described herein are implemented in concert with a controller that comprises a microprocessor executing control instructions. The controller can be in the form of any computing device, including desktop, laptop, and mobile device, and can communicate control signals to the measurement devices in wired or wireless form.

The present application discloses an appropriately sized rectangular radio frequency (RF) resonant cavity to optimally measure the resonant characteristics of a biological body. The RCP technique employed in the present specification uses a non-ionizing radiofrequency for making periodic measurements of body composition which may be used as an indicator of lifelong metabolic health. Further, RCP is sensitive to changes in composition, making it an accurate and repeatable marker of metabolic health. Specifically, RCP uses radiofrequency to measure the water content of the biological body inside the resonant cavity. The biological body, in various embodiments, comprises in vivo children, in vivo adults, in vivo animals, cadaver animals, and meat products. Composition of meat products is beneficial for analyzing samples in the met industry.

Resonant cavities are closed, conductive structures which confine electromagnetic waves of distinct frequencies to the interior of the resonant cavity. Electromagnetic waves bounce back and forth between the inside walls of the resonant cavity to form resonant modes of distinct frequencies. Some of these frequencies destructively interfere and vanish while others constructively interfere and reinforce each other to form standing waves with frequencies corresponding to the geometry of the system. In various embodiments, shifts to the resonant properties can be made by inserting objects that change the average dielectric properties of the cavity, by distorting the shape of the cavity, or any other change which disturbs the fundamental geometry of the system. As further discussed below, measurements and analyses of these shifts can provide insight into the composition of the biological object causing the changes to the dielectric properties of the cavity.

In a known experiment for measuring the water content of two adult males before and after drinking 2 L of water, it was observed that the RCP technique is sensitive enough to detect a 2% change in total body water in adult males. This is similar in sensitivity to DXA and more sensitive to total body water than ADP, thus the RCP method is sensitive enough to measure changes in body composition. Further, fat mass may be accurately measured in a spherical phantom of unknown composition by using a resonant cavity. In a study which took a systematic look at the accuracy of measuring body composition using a resonant cavity, the resonant cavity was characterized using phantom objects of varying, but known, composition and volume to build a reference catalog for measuring body composition in unknown samples. Using the phantom calibration, a spherical phantom of known volume and unknown composition was measured using the RCP technique and found to be within 2% of its actual fat mass. In embodiments, the method of the disclosed RCP method characterizes a resonant cavity to accurately measure body composition in arbitrarily shaped phantoms, which will allow the translation of the findings more directly to humans. In an embodiment, the present specification provides an RCP cavity which is sized for pediatric use in a clinic.

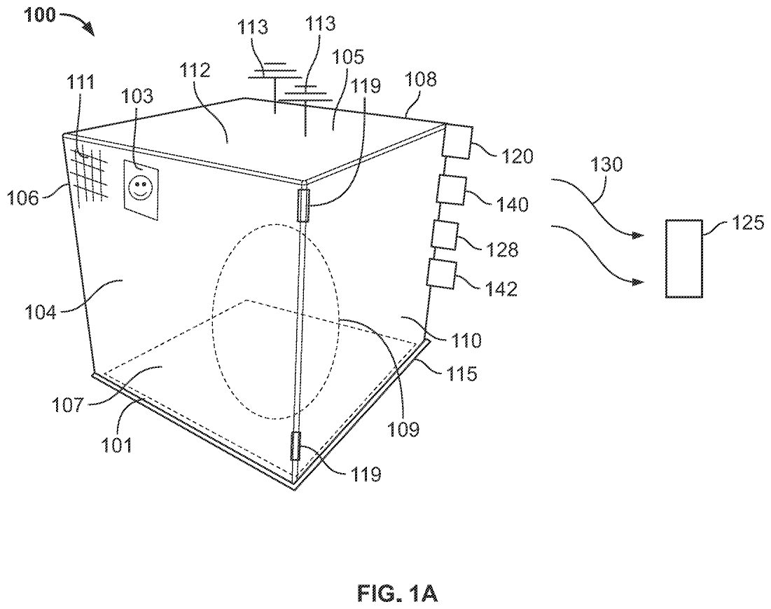

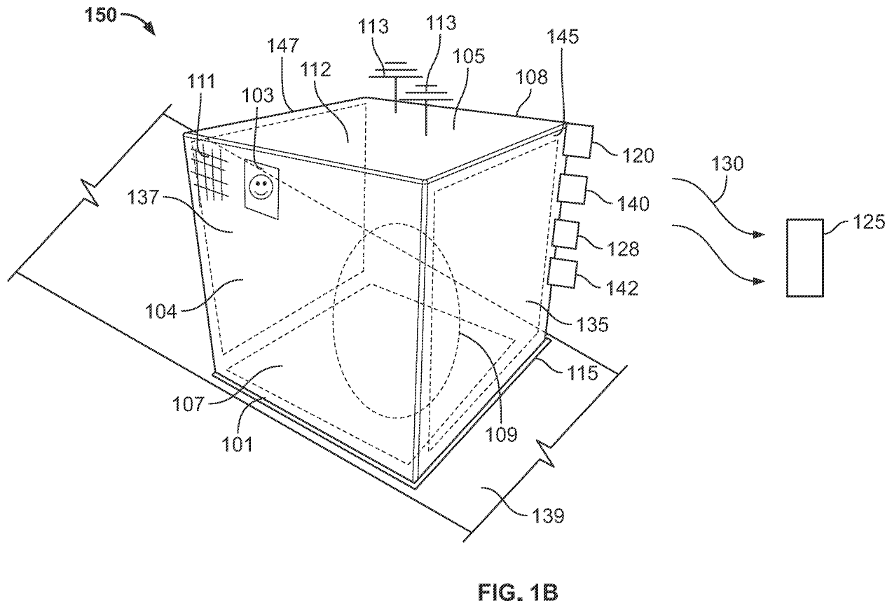

FIG. 1A is an illustration of a resonant cavity perturbation (RCP) measurement system 100 in accordance with one embodiment of the present specification. FIG. 1B is an illustration of an integrated resonant cavity perturbation (RCP) measurement system 150 in accordance with another embodiment of the present specification. Referring to FIG. 1A, the integrated resonant cavity perturbation (RCP) measurement system 100 comprises a box-like resonant cavity structure 105, having a base 101, four sides 104, 106, 108, 110, and a top 112. In one embodiment, side 110 is transparent, is see-through, or otherwise comprises material that permits light to pass through. In the embodiment depicted in FIG. 1A, the side 110 is attached to the rest of the resonant cavity structure 105 with hinges 119 to thereby function as a door or portal through which an animal body may be positioned in the internal space of the resonant cavity structure 105. Referring to FIG. 1B, the integrated resonant cavity perturbation (RCP) measurement system 150 comprises a tunnel-like resonant cavity structure 105, having a base 101, two sides 104, 108, and a top 112. A first opening 135 is located on a first side of the resonant cavity structure 105 and a second opening 137 is located on a second side of the resonant cavity structure 105, opposite the first side. A conveyor belt 139 is positioned under the resonant cavity structure 105 and is configured to convey a biological body 109 through the resonant cavity structure 105 into the first side 135, into the structure 105, and out of the second side 137, for measurements. While the biological body 109 is within the structure 105, two additional sides 145, 147 are manually or automatically placed over first and second openings 135, 137 respectively, to fully enclose the biological body for measuring.

Referring to FIGS. 1A and 1B simultaneously, physically integrated into, or coupled to, the base of the resonant cavity structure 105 is a scale 115 configured to measure the weight of a biological body 109 positioned in the resonant cavity structure 105. In embodiments, physically integrated into, or coupled to, at least one of the sides, is a height measurement system 120 which may comprise a camera or optical device configured to detect the top of a biological body 109 positioned in the resonant cavity structure 105 and derive a height measurement. In embodiments, the system 100 includes a motion tracking device 142 configured to measure a shape of the biological body. In the embodiment shown in FIG. 1A, the motion tracking device 142 is attached to the transparent side 110. Data 130 indicative of the biological body's composition, height, and weight are transmitted by a transceiver 128 in the integrated measurement system 100 and received by a controller 125 which may be wirelessly connected or in wired communication with the integrated measurement system 100.

In embodiments, the resonant cavity structure 105 comprises a wood, plastic, or cardboard frame with a metal sheet or mesh 111, such as copper, lining the walls thereof. In some embodiments, the resonant cavity structure 105 comprises a non-conductive veneer 103 to create a more friendly, comforting environment into which infants, toddlers, and children will be more likely to actively enter and remain. In embodiments, the veneer 103 comprises a non-conductive plastic or fabric. Though only partially shown in FIGS. 1A and 1B, the mesh 111 covers the entirety of the base 101, four sides 104, 106, 108, 110 in FIG. 1A and four sides 104, 108, 145, 147 in FIG. 1B, and the top 112 of the resonant cavity structure 105 and is only shown partially to allow visualization of the remainder of the structure 105. In embodiments, the mesh 111 comprises an inner-most layer of each of the base, sides, and top. Though only partially shown in FIGS. 1A and 1B, in various embodiments, the veneer 103 covers only a portion of or the entirety of the base 101, four sides 104, 106, 108, 110 in FIG. 1A and four sides 104, 108, 145, 147 in FIG. 1B, and the top 112 of the resonant cavity structure 105 and is only shown partially to allow visualization of the remainder of the structure 105. In one embodiment, the resonant cavity structure 105 comprises a rigid wooden frame with copper mesh pulled tightly onto the structure. Ideally, the resonant cavity structure 105 would have perfectly conducting walls, such as solid sheets of copper, and have broad-band antennae. Preferably all seams are electromagnetically sealed using, for example, copper tape with conductive adhesive applied to the corners and along any seams. Inside the resonant cavity structure 105 is a pad 107, outline, marking or label designating where a body should be placed. In various embodiments, the resonant cavity structure 105 has dimensions in a range of 0.2 m×0.4 m×0.1 m to 6.0 m×5 m×3 m. In one embodiment, the resonant cavity structure 105 has dimensions greater than 1.0 m×1.5 m×0.4 m and is configured to accommodate biological bodies ranging from infants to cattle. In one embodiment, the resonant cavity structure 105 has dimensions of 3 m×2.25 m×1.125 m. In some embodiments, the resonant cavity structure 105 is sized similarly to a phone booth or small shower. In another embodiment, the resonant cavity structure 105 is configured to accommodate biological bodies having a volume of at least 5 L. In various embodiments, the size of the resonant cavity structure may be adjusted to accommodate larger or smaller biological samples, for example, humans or livestock.

At least two antennae 113, and, in embodiments, preferably 4 to 6 antennae, are used to detect and measure one or more resonant modes in the resonant cavity structure 105, with each pair of antennae tuned to a specific direction in coordinate space. A first antenna of the pair transmits a signal, while the second receives the signal. Where there are two pairs of antennae, preferably each pair is tuned to different directions, each pair orthogonal to another. For example, a first pair is directed along the x axis while the second pair is directed along the y axis. Where there are three pairs of antennae, preferably each pair tuned to different directions, each pair orthogonal to the others, such that a first pair is directed along the x axis, a second pair is directed along the y axis, and a third pair is directed along the z axis. In embodiments, a single pair of antennae measures the electrical scattering parameter (S-parameters) S21, which is the power transferred from a first port to a second port. Using a pair of antennae reduces noise and error in measurements. In other embodiments, the resonant cavity structure includes only a single antenna, and the S11 or S12 parameter is measured using the single antenna.

Resonant cavities have two measurable properties of interest: resonant frequency and quality factor (Q-factor). In one embodiment, the resonant cavity is designed to have a predefined baseline resonant frequency, such as 180.15 MHz. In embodiments, the baseline resonant frequency will be unique to each cavity because it is dependent on the physical dimensions of the cavity. The Q-factor is a measured ratio of the energy stored in the cavity to the amount of energy applied to it. Q-factor may be affected by physical defects, material quality, and other factors that make it incalculable. These two properties, resonant frequency and Q-factor, are measured using a network analyzer 140 that is in wired communication with the interior of the resonant cavity structure 105. The network analyzer 140 measures the scattering parameters (s-parameters). S-parameters describe the way an electric signal changes when incident on a discontinuity and are typically measured in the RF or microwave regimes. The S-parameters form a square matrix based on the number of available ports. In one embodiment, the network analyzer 140 configured to collect data has two ports, resulting in a 2×2 S-parameter matrix.

In one embodiment, the network analyzer is a scalar network analyzer (SNA) or a vector network analyzer (VNA). An SNA exclusively measures the amplitude properties and is functionally equivalent to a spectrum analyzer. On the other hand, VNAs measure both the amplitude and phase properties of an electric signal and may be used to measure RF and microwave cavities.

In an embodiment, a vector network analyzer (VNA) and an oscillator/sweep signal are coupled with a resonant cavity, wherein the VNA and the oscillator are combined into a single device. Inside the resonant cavity, electromagnetic waves propagate back and forth between the conductive walls of the cavity. Some of these frequencies destructively interfere and vanish, while others constructively interfere and reinforce each other to form standing waves of discrete frequencies corresponding to the geometry of the system. These discrete frequencies are referred to as resonant modes or transverse electric (TEₘₙₚ) modes. These TEₘₙₚ modes are defined by the indices m, n, and p which take on integer values from zero on and associate the mode with the x, y, and z directions respectively. The TEₘₙₚ modes take the form of equation:

$${f}_{mnp} = \frac{c}{2} \sqrt{{( \frac{m}{w} )}^{2} + {( \frac{n}{l} )}^{2} + {( \frac{p}{h} )}^{2}}$$

where w is the width of the cavity, l is the length of the cavity, h is the height of the cavity, and fₘₙₚ is the TEₘₙₚ resonant frequency. The RCP technique implies a perturbation or small change to the system which will alter the resonant mode and includes, but is not limited to, adding a dielectric sample or biological body interior to the cavity. Dielectric constants of these materials are complex, comprising ∈*=∈′−i∈″, where ∈′ is the relative dielectric constant and ∈″ is the relative loss factor. Introducing a perturbation to the system alters the resonant mode as:

$$Δ f = - 2 {f}_{0} {K}_{sh} ( {ϵ}^{′} - 1 ) \frac{{V}_{s}}{{V}_{c}}$$

where Δf is the shift in resonant frequency, f₀ is the resonant frequency of the empty cavity, Kₛₕ is the shape factor of the sample, Vₛ is the volume of the sample, and V_{c} is the volume of the cavity. The shifts in the resonant frequency are measureable and predictable, which allow for an accurate measurement of total body water and body composition.

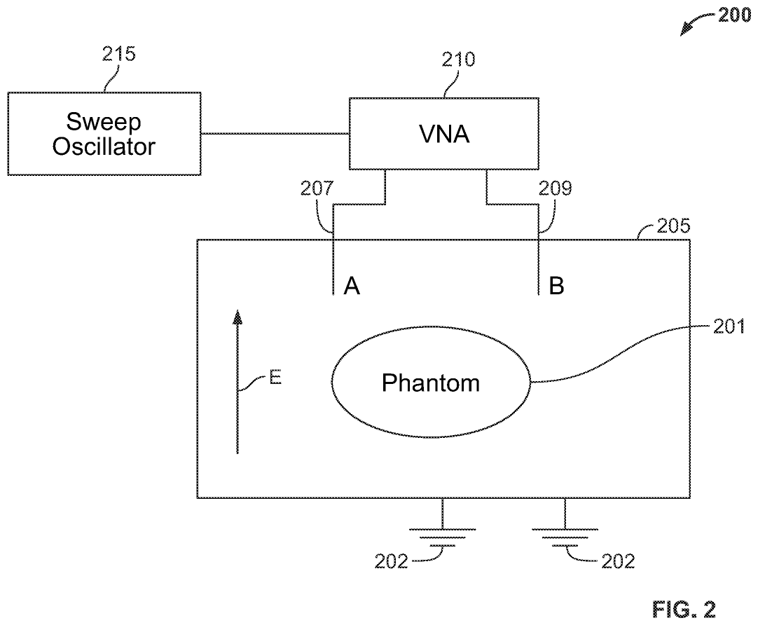

FIG. 2 illustrates an integrated resonant cavity perturbation (RCP) measurement system 200, in accordance with an embodiment of the present specification. System 200 comprises a cavity 205, a vector network analyzer (VNA) 210, an oscillator 215 and two ground plane antennae 202. System 200 represents a two-port network wherein a first port is labelled as Port A 207 and a second port is labelled as Port B 209. The ports 207, 209 are used to measure scattering parameters. In some embodiments, more than two ports are included in RCP measurement system. In one embodiment, four ports are used in an RCP measurement system. In one embodiment, referring to FIG. 2, the network analyzer VNA 210 is configured to measure the S21 parameter for the cavity 205. The S21 parameter measures the change in the electric field between the two ground-plane antennae 202. In embodiments, the antennae 202 are variable antennae and resonate at a frequency different than a resonant frequency of the cavity. In some embodiments, the antennae 202 are quarter-wavelength antennae. The cavity's 205 resonant properties are measured using the VNA 210 which is in data communication with the sweep oscillator 215.

The method of the present specification, measures body composition by placing an object/sample/phantom 201 inside the cavity to change the resonant properties, as shown in FIG. 2. The RCP method is sensitive to the shape and volume of the sample (Vₛₐₘₚₗₑ), and comprises placing a phantom sample 201 having a volume smaller than the volume of the cavity (V_{cavity}), resulting in a small fill factor (F), wherein: F=(Vₛₐₘₚₗₑ/V_{cavity})<0.005. The dielectric properties and volume of the phantom sample measurably affect the resonant cavity. As the volume of the sample increases, the magnitude of the fill factor increases and begins to affect the resonant properties of the cavity. Resonant cavity perturbation begins to behave differently when the perturbation is no longer small. In an embodiment, the system and method of the present specification provide a resonant cavity of a size and shape that can comfortably accommodate a human body for accurate measurement of body composition even with greater fill factors than F<0.005. In an embodiment, the resonant cavity has dimensions of 3 m×2.25 m×1.125 m to accommodate a human body and ensure that the interior walls of the system are positioned at least one foot away from a person positioned within the cavity. In embodiments, the system and method of the present specification may be used to accurately measure fat mass in phantoms of arbitrary distribution and composition. In an embodiment, phantoms having a same mass and composition, but different mass distributions are indistinguishable.

In one embodiment, the measurement system 200 uses the S-parameter measurements which compare the energy of the reference signal to the energy of transferred signal. The S-parameters are measured by comparing the reference signal produced by Port A 207 to the signal received by Port B 209, which allows for the direct measurement of the S12 and S21 parameters. The S21 parameter is directly measured throughout data collection and the raw data is saved locally or transmitted to a storage unit or memory. Prior to doing so, the system is preferably calibrated by, for example, taking a baseline measurement of the surrounding room using the VNA 210, comparing it to the measurement of the inside of the sealed cavity 205, establishing that the cavity 205 is electromagnetically isolated, and determining the baseline resonant properties of the cavity 205.

A collection of standing waves within the resonant cavity 205 is known as “mode” and takes the form of TE_{xyz}. In one embodiment, the system 200 is configured to repeatedly perform a measurement of a vertical mode inside of the resonant cavity 205 and use the VNA 210 to measure the S21 parameter of this mode. The full range of frequencies which are swept throughout the cavity are collectively known as an electrical network. In embodiments, the VNA 210 measures parameters of electrical networks for characterizing a two-port network. The scattering parameter, or S-parameter, is measured to show the network differences between the two ports. In an embodiment, a Gnuplot is used to measure the S21 parameter of the TE₁₁₀ mode inside the resonant cavity 205. In an embodiment, a fitting routine is used for analyzing the TE₁₁₀ mode measured using the S21 parameter of the VNA 210 for the resonant cavity 205. The resonant cavity 205 used in said embodiment measures 1.0 m×1.5 m×0.4 m and is expected to resonate near 173.5 MHz. In embodiments an Array Solutions Ultra High Frequency Network Analyzer is used to measure scattering parameters (S-parameters) of electrical network responses, the scattering parameters compare the impedance measured between ports. The Array Solutions VNA UHF covers a range between 5 kHz and 1 GHz, and has two ports for operational use. A sweep signal generator is attached to port 1, while impedance and phase measurements are available from both ports. The sweep signal generator produces a signal of constant amplitude and varying frequency, allowing for frequency-dependent system responses to be measured.

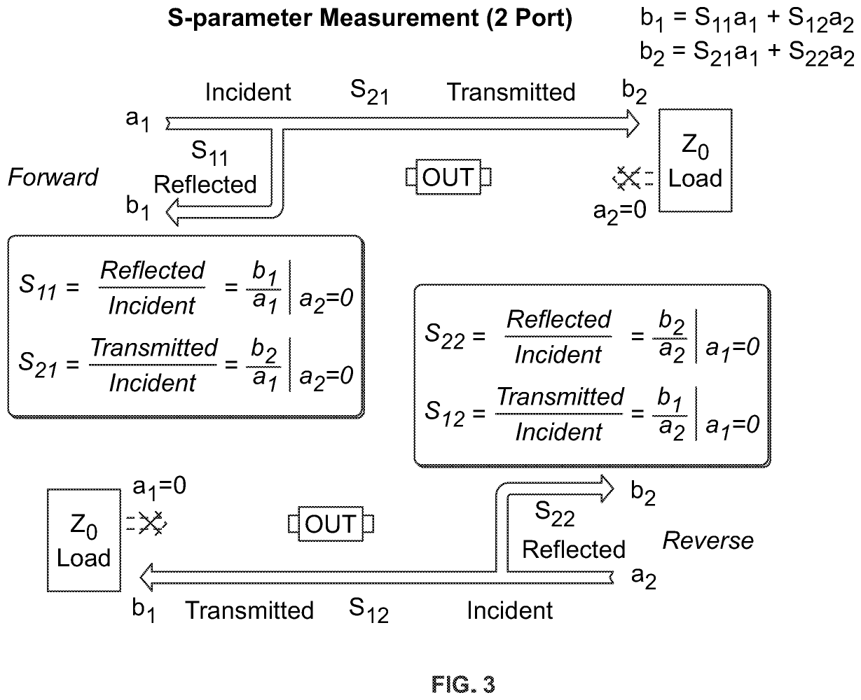

FIG. 3 is a schematic diagram of the scattering parameters of a network analyzer, in accordance with an embodiment of the present specification. Referring to FIGS. 2 and 3, in general, the S-parameter of an n-port system have n² coefficients and form a symmetrical n×n matrix. In embodiments, the VNA 210 used for data acquisition has two distinct ports, Port A 207 and Port B 209, resulting in a 2×2 scattering matrix of the form:

$$S = ( \begin{matrix}{S}_{11} & {S}_{12} \\ {S}_{21} & {S}_{22}\end{matrix} )$$

where the entries Sᵢⱼ indicate the S-parameter between ports i and j. For measurement of the TE₁₁₀ mode we are most interested in the S21 parameter, which compares the sweep signal produced in Port B 209 to the signal received after passing through the resonant cavity in Port B 209. In an embodiment, in order to determine the resonant characteristics of the cavity 205, the antenna 202 are placed at equal distances from the sides of the cavity 205 and equidistant to each other. One antenna is attached to the sweep oscillator 215 at Port A 207, while the second antenna is attached to Port B 209 to measure the S21 parameter.

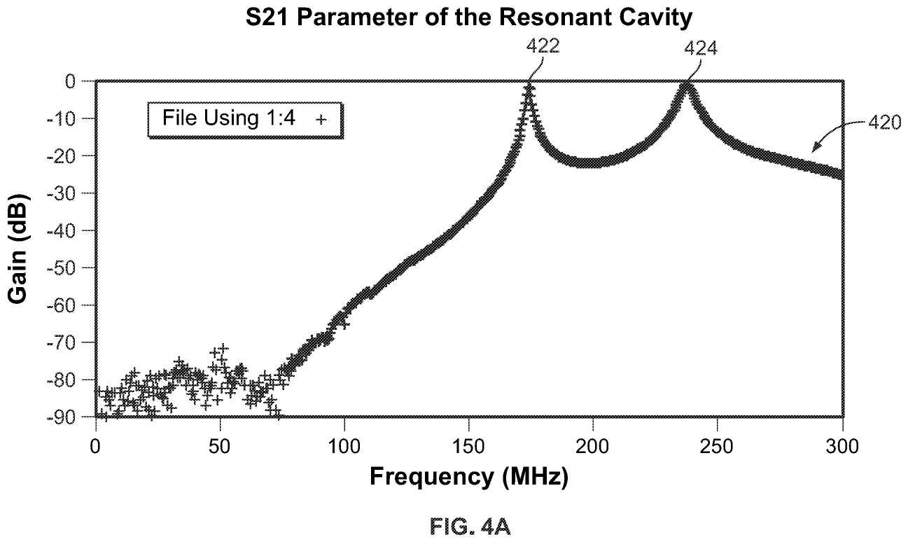

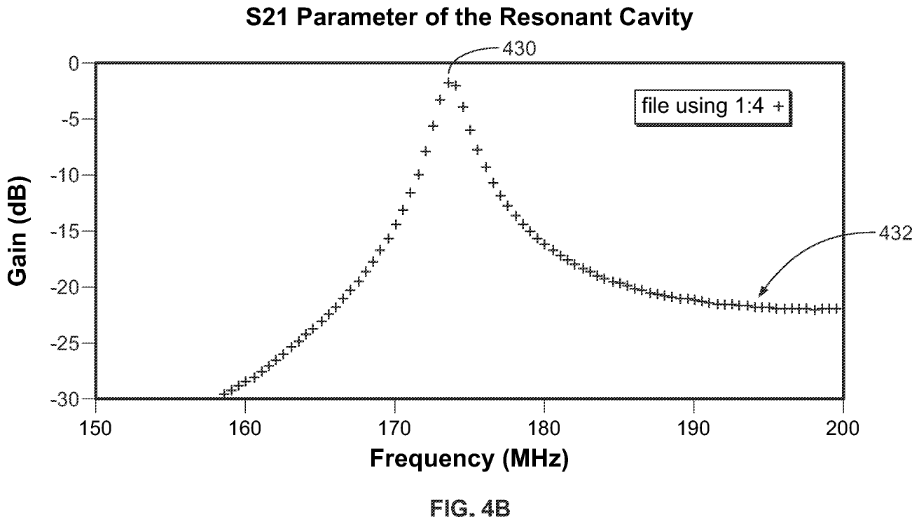

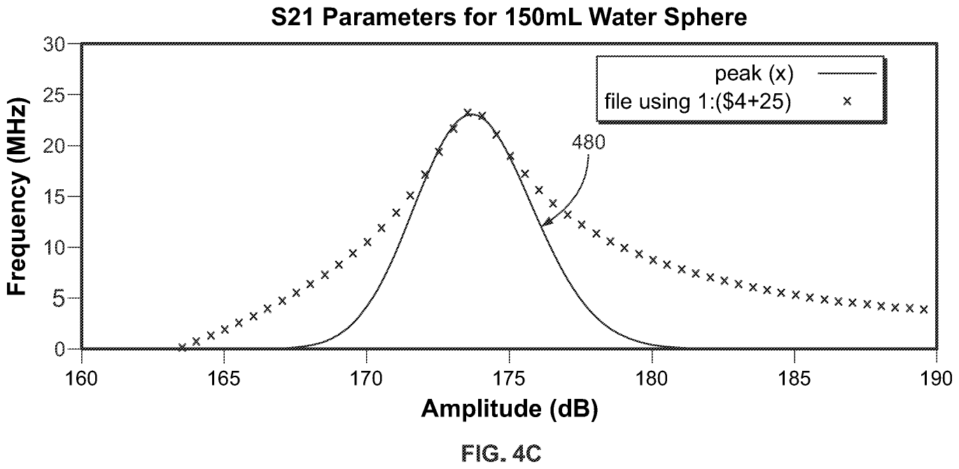

FIG. 4A illustrates a response of the resonant characteristics of the resonant cavity, in accordance with an embodiment of the present specification. Referring to FIG. 4A, as can be seen from graph 420, a peak 422 represents the TE₁₁₀ mode which occurs close to a network frequency of 170 MHz; and a second peak 424 represents the TE₁₂₀ mode which occurs close to a network frequency of 240 MHz. Both modes form narrow peaks 422, 424 respectively, around their resonant frequency, however there is a lot of noise between the two resonant peaks 422, 424. For analysis, in an embodiment, the data is adjusted to be positive by adding 25 dB to all the data. This does not change the overall calculations, but allows a Gnuplot to more easily identify the peaks 422, 424 representing the TE₁₁₀ and TE₁₂₀ modes respectively.

Referring to FIGS. 2, 4A, and 4B, in an embodiment, a Skew-Gaussian fit is applied to these peaks 422, 424 to measure the response of the system 200. FIG. 4B illustrates a Gaussian-like distribution 432 for the S21 parameter of the resonant cavity 205 in TE₁₁₀ mode, in accordance with an embodiment of the present specification. Referring to FIGS. 2 and 4B, the peak 430 of the distribution 432 is the resonant frequency of the TE₁₁₀ mode, and the width of the distribution corresponds to Q-factor of the data. As is known, Q-factor, or quality factor, is a measurement of how much energy is lost into the system. A high Q-factor corresponds to a lower energy loss, and means the system is better suited for measuring body composition.

As is known, the general form of a Gaussian distribution is:

$$G ( x ) = \frac{A}{\sqrt{2 {πσ}^{2}}} exp ( - \frac{{( x - μ )}^{2}}{2 {σ}^{2}} )$$

where A is the amplitude, σ is the standard deviation, σ² is the variance, and μ is the central value or mean of the distribution. Gaussian distributions form narrow, symmetric peaks which closely resemble the distribution for the TE₁₁₀ mode but there is a slight asymmetry between the leading edge and trailing edge of the distribution. This asymmetry can be accounted for by applying a skew Gaussian fit. Skew Gaussian fits combines a Gaussian distribution with a cumulative distribution function (CDF) in order to account for variance throughout a dataset. The two functions are multiplied together to form the Skew gaussian fit:

$$S ( x ) = G ( x ) * ( 1 + erf ( \frac{x - μ}{σ \sqrt{2}} ) ) .$$

Any software may be used for generating a fitting routine. As an example, Gnuplot is a graphing software which can be downloaded for use on any computer running Windows 10 (or other compatible OS) and may be used to develop this fitting routine.

FIG. 4C is a graph illustrating a peak-fit and data for the TE₁₁₀ mode of the resonant cavity obtained by using a Gnuplot fitting routine, in accordance with an embodiment of the present specification. As can be seen from plot 480, by using Gnuplot the measured central frequency is 172.3887±0.04131 MHz and the standard deviation is 2.53436±0.02313 MHz. The measured values enable determination of the resonant properties of the resonant cavity, wherein the Q-factor for a resonant cavity may be depicted as:

$$Q = 2 π \frac{{f}_{0} E}{P} = 2 π \frac{{f}_{0}}{σ}$$

where E is the magnitude of the electric field and P is the power in Watts of the electric field.

Referring to FIG. 2, in an embodiment, data collected using the VNA 210 is saved in a data format, such as a CSV file, and later processed using, for example, a Laplace distribution fitting routine executed by a processor in a computing device. The Laplace distribution is the distribution of the differences between two identical exponential distributions. In an embodiment, the difference between exponential distribution from Port A 207 and the exponential distribution from Port B 209 has a probability density function given by the equation:

$$S ( x ) = \frac{A}{\sqrt{2 {πσ}^{2}}} exp ( - \frac{{( x - {f}_{0} )}^{2}}{2 {σ}^{2}} ) * ( 1 + erf ( \frac{x - {f}_{0}}{σ \sqrt{2}} ) ) .$$

where x is the frequency, A is the amplitude of the resoanance, f₀ is the resonant frequency of the empty cavity, and σ is the full width half max which correlates to the Q-factor. It should be appreciated that there are two independent quantities which define the resonant properties of the cavity: the resonant frequency and the Q-factor. Both quantities possess an exponential distribution, thus the Laplace distribution provides an appropriate means to analyze information from the VNA.

In various embodiments, the VNA 210 is configured to measure a broad spectrum between 5 kHz and 1200 MHz. In one embodiment, the analysis performed by the system focuses on a primary vertical mode and uses a truncated range of measurement, namely between 5 kHz and 300 MHz. In one embodiment, only a vertical mode is measured and used to perform the analyses described herein.

In an embodiment, a two-compartment model of body composition is used, dividing the body into fat and lean masses. A resonant cavity may be characterized to accurately measure fat mass in spherical samples by creating calibration curves for samples of known composition and extrapolating to determine the fat mass of an unknown sample within 2% of its actual fat mass. Since, bone is primarily composed of minerals and water, which have a negligent effect on resonant properties, said model provides an accurate method for characterizing RCP technology for measuring infant body composition. In an embodiment, a phantom dielectric sample/object is constructed from biologically equivalent materials to characterize that the RCP technology is suitable for human body composition measurements. In the construction, lean mass is modeled with water and fat mass is modeled with culinary lard. The phantom dielectric object may be made in various shapes, such as but not limited to, spheres, rectangles and irregular shapes obtained by mixing combining lard and water into an emulsion by using a negligible quantity of dish soap and distributing said emulsion into plastic containers to create electromagnetically body equivalent phantoms. In embodiments, the phantom object made of lard and water is contained in plastic containers as said containers are invisible to the VNA at the predefined frequencies, and does not affect the resonant properties of the phantom object.

As is known, fat mass distribution affects the shape of a sample/phantom object and thereby may affect the body composition measurement by using the RCP technique. The ability of RCP technique to measure body composition has been limited using known methods. Samples have been limited to being simple shapes, uniform distribution and small in volume relative to the volume of the resonant cavity being used. Known studies have investigated phantoms of uniform composition. However, a human body is not made of a uniform mixture of lean and fat masses.

In various embodiments, phantom dielectric object of equivalent mass but different shapes provide identical body composition measurement results. As is known, a collection of standing waves within a resonant cavity is known as “mode” and takes the form of TE_{xyz}. In an embodiment, the method of the present specification uses the Kraszewski method which measures multiple primary resonant modes and averages said measurements to reduce the influence of shape on shifts in resonant properties. In embodiments, primary resonant modes include TE110, TE101, and TE011 and secondary resonant modes include TE210, TE120, TE012, and TE021. In embodiments the system of the present specification comprises a rectangular resonant cavity designed to have three unique primary modes, being: a primary vertical mode, a primary horizontal mode and a primary transverse mode. Resonant cavities are sensitive to changes in geometry and or composition and rely on the accurate measurement of their resonant modes. In embodiments, a first antenna coupled with the cavity measures the primary vertical mode, and two additional antennae may be attached with the cavity to measure the primary horizontal mode and the primary transverse mode. In various embodiments, combining the data collected by operating the cavity in the three modes minimizes the influence of shape on shifts in resonant properties. In other embodiments, resonant cavities with multiple primary modes are used to increase the accuracy of measurement of body composition independently of the body shape. In an embodiment, the method of the present specification uses the data collected by operating the resonant cavity in at least the three primary modes to form calibration curves of samples/phantoms with known shapes, compositions, distribution and mass to characterize the cavity along the three primary modes.

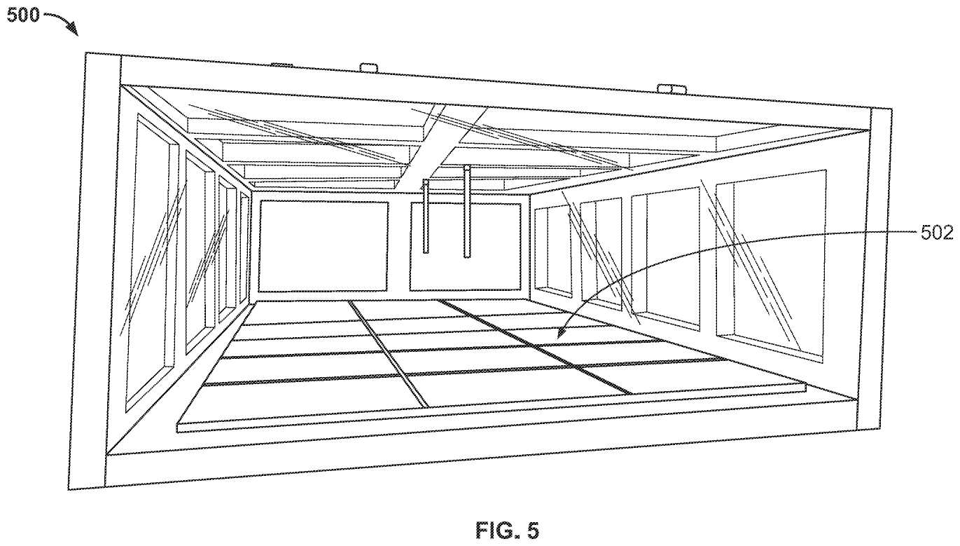

FIG. 5 illustrates an exemplary resonant cavity, in accordance with an embodiment of the present specification. An interior region 502 of resonant cavity 500 can be accessed via access doors (not shown in the figure). In various embodiments, the present specification provides an RCP device for clinical measurements, which is capable of accommodating human sized samples, is comfortable for patients and of a size that is suitable for use in a clinic. In an embodiment, the RCP device of the present specification is suitably sized for measuring the body composition of infants and children.

With reference to FIG. 2, in one embodiment, the resonant cavity 205 has a baseline resonant frequency of (173.73±0.48) MHz and a Q-factor of (451.21±8.38). The analytical technique disclosed herein uses the difference between the baseline resonant frequency and the resonant frequency of the cavity 205 with a dielectric sample 201 placed inside. Based on the difference, a determination of body composition, namely the extent of fat, muscle and/or non-fat material in a body, may be achieved. More specifically, the presently disclosed system accurately measures the fat mass of a sample, i.e. a biological body, within 2% of known values. Accordingly, the screened enclosures described herein may act as an RF resonant cavity designed that is sensitive to, and reflective of, the lean body mass of infants or other animal bodies.

In one embodiment, the system corrects for shape. Shape factors may be analytically calculated for sufficiently simple shapes such cylinders, spheres, or boxes. In embodiments, odd or complex shapes require either experimental measurement or approximation using simple shapes. In another embodiment, the system corrects for shape but does not correct for salinity because it has been determined that there is no effect of the biological range of salinity on resonant shifts.

Data gathered by each of the one or more antennae are stored in a local or remote memory and then processed by a processor. The processor in the integrated measurement systems of the present specification are configured to analyze the raw data generated by the resonant cavity structure and apply a plurality of programmatic analyses to determine a body composition.

An exemplary programmatic analyses which includes the application of certain boundary conditions and analytical modes is provided below:

%% Example Fitting routine for MatLab analysis environment. This routine %%
focuses on a Laplacian fit, which is commonly referred to as a modified %%
or skew Gaussian fit. It is characterized by the standard exponential %%
fit of a Gaussian, but offset by the error function in order to allow %%
for imperfections in the system which cause asymmetries in the data.
%% Data collected using an Array Solutions VNA UHF which measures the S21
%% parameter of a resonant cavity. The magnitude of the S21 parameter and
%% the frequency range are recorded in a .csv file.
%% set up workspace
clf; close all; clc; clear all;

| | |
|---|---|
| format compact | % Suppresses blank lines in output |
| global c | % defines a global variable |

%% Importing Data
% load file (s)
importdata(‘042817_1100mL_50emulsion_1_B.csv’);
M1 = csvread(‘042817_1100mL_50emulsion_1_B.csv’,5,0);
%% Sort the Raw Data
% Extract frequency range and S21 magnitude from data file

| | | |
|---|---|---|
| M1_f_raw | = M1(:,1); | % Frequency |
| M1_S21_mag_raw | = M1(:,4); | % S21 mag (dB) |

| | | |
|---|---|---|
| M1_w_raw | = 2*pi*M1_f_raw; | % angular frequency |

%% Fitting the Data Measurement 1
% Cleaning the Data

| | |
|---|---|
| M1_data_raw = [M1_w_raw,M1_S21_mag_raw]; | % reformatting data % |

into a 2 column data set

| | |
|---|---|
| M1_data = M1_data_raw(M1_data_raw(:,1)>700,:); | % cleaning data, |

exclude the noise for w < 700

| | |
|---|---|
| M1_w = M1_data(:,1); | % cleaned frequency |

data

| | |
|---|---|
| M1_S21_mag = M1_data(:,2); | % cleaned S21 |

parameter data
% Create a two lorentzian peaks and add random noise
M1_k = 2; % step size along x-axis; smaller k = more points in signal
M1_t = [−500:M1_k:700];
M1_Baseline = 0.05;
M1_a = 0.05;
M1_y = M1_S21_mag’;
M1_t = M1_w;
% Perform an iterative fit using the FMINSEARCH function
% start=[length(t)/3 length(t)/2 length(t)/1.5 length(t)/2];

| | |
|---|---|
| start = [1050 30 1450 30]; | % [center1 maxloss center2 |

maxloss]
% start now has guesses for BOTH peaks

| | |
|---|---|
| options = optimset(‘TolX’,0.1); | % Determines how close the |

model must fit the data
% “tic” and “toc” measure how long the iterative fit takes.
tic
M1_parameter =
fminsearch(@(lambda)(fitlaplacianb(lambda,M1_t,M1_y)),start);
toc
M1_Baseline = c(1)
M1_Measured1 = [c(2) M1_parameter(1) M1_parameter(2)] % Peak height 1 is
returned in the global variable c(1).
M1_Measured2 = [c(3) M1_parameter(3) M1_parameter(4)] % Peak height 2 is
returned in the global variable c(2).
M1_slope = c(4)
M1_slope2 = c(5)
% Compute a model and plot it (blue line) along with
% the original data (red points)
M1_peak1 = c(2).*laplacian(M1_t,M1_parameter(1),M1_parameter(2));
M1_peak2 = c(3).*laplacian(M1_t,M1_parameter(3),M1_parameter(4));
M1_model = M1_Baseline + M1_peak1 + M1_peak2 + M1_slope*M1_t +
M1_slope2*M1_t.\hat{ }2;
M1_background = M1_Baseline + M1_slope*M1_w + M1_slope2*M1_w.\hat{ }2;
M1_mag1_corr = M1_S21_mag − M1_background;
%% resonant frequencies
M1_f110 = M1_Measured1(2)/(2*pi)
%% Q-factors
M1_Q110 = 1000*M1_Measured1(1)/(2*pi)
%% frequency shifts: TE 110
% shift_M1_f110 = 173.4278 − M1_f110
% %% Q-factor shifts: TE110
% shift_M1_Q110 = 452.6097 − M1_Q110
%% Graphs/Figures
figure(1);
subplot(2,2,[1,2]);
plot(M1_f_raw,M1_S21_mag_raw,‘*’);
title(‘1000g RO water Supine: Trial 1’);
xlabel(‘frequency (MHz)’);ylabel(‘loss (dB)’);
subplot(2,2,3);
plot(M1_t,M1_y,‘r.’,M1_t,M1_peak1,‘c’,M1_t,M1_peak2,‘m’,M1_t,M1_model,‘b’,
M1_t,M1_background,‘k’ );
title(‘Laplacian fits of the S21 parameter’);
xlabel(‘w (2\piHz)’);ylabel(‘loss (dB)’);
subplot(2,2,4);
plot(M1_w,M1_mag1_corr,‘b.’); M1_a = 1;
title(‘Resonant Peaks neglecting background noise’);
xlabel(‘\omega’);ylabel(‘loss (dB)’);

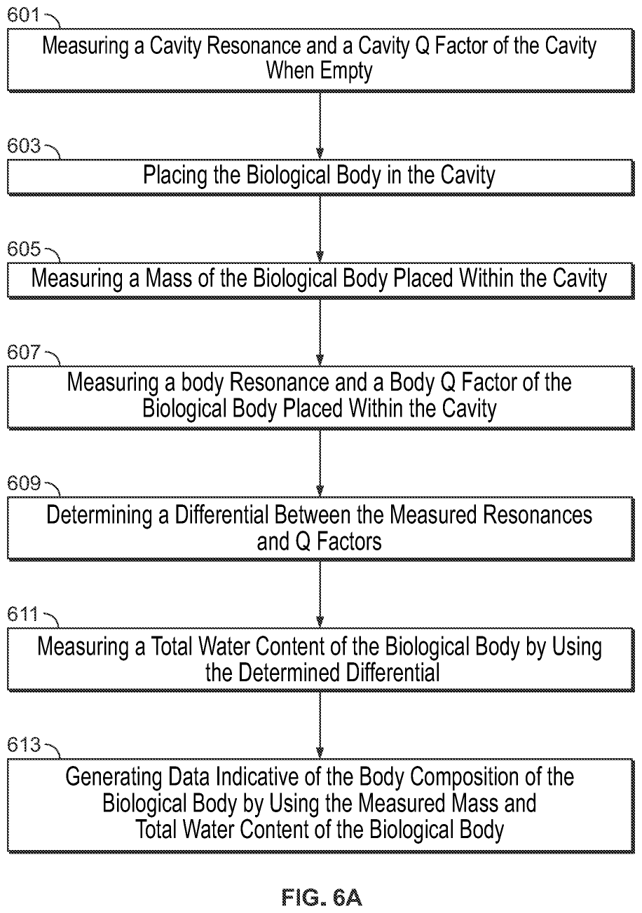

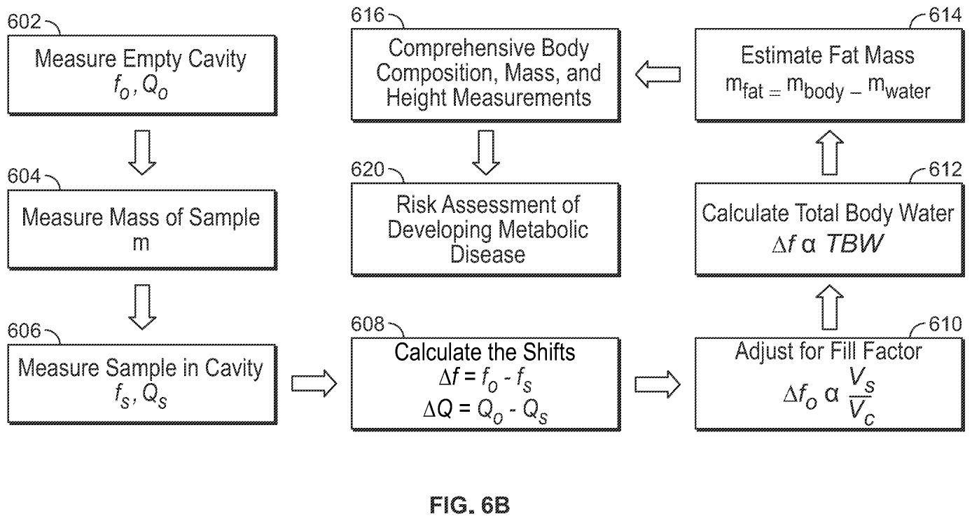

FIG. 6A is a flowchart illustrating a method for generating a body composition measurement of a biological body, by using a resonant cavity comprising a frame and metal material lining the frame, in accordance with an embodiment of the present specification. In embodiments, the resonant cavity has a top panel, bottom panel, and four side panels to thereby define an enclosed volume; at least one antenna; a network analyzer in data communication with the at least one antenna; and a controller configured to receive data from the network analyzer and generate an output. At step 601 a cavity resonance and a cavity Q factor of the resonant cavity are measured before placing the biological body in the cavity, i.e. when the resonant cavity empty. At step 603, the biological body is placed in the cavity. At step 605 mass of the biological body placed within the cavity is measured. At step 607 a body resonance and a body Q factor of the biological body placed within the cavity is measured. At step 609 a differential between the measured resonances and Q factors of the empty cavity and the body placed within the cavity is determined. At step 611 a total water content of the biological body is determined by using the determined differential; and at step 613 data indicative of the body composition of the biological body is generated by using the measured mass and total water content of the biological body. The method depicted in FIG. 6A is further described in detail with reference to FIG. 6B.

FIG. 6B is a flowchart illustrating the steps in a method of using an integrated resonant cavity perturbation (RCP) measurement system to measure body composition and provide a risk assessment of developing metabolic disease, in accordance with an embodiment of the present specification. At step 602, the resonance f₀ and Q factor Q₀ of an empty cavity are measured. In some embodiments, for larger fill factors, these values are determined using an empirically determined non-linear correction factor. At step 604 the mass of a sample m in the cavity is measured. Then, at step 606, the resonance fₛ and Q factor Qₛ of the sample are measured. The shifts are calculated at step 608, using the equation Δf=f₀−fₛ for the resonance shift and ΔQ=Q₀−Qₛ for the Q factor shift. At step 610, the fill factor is adjusted using the formula Δf₀∝Vₛ/V_{c} where Vₛ is the volume of the sample and V_{c} is the volume of the cavity. Total body water is calculated at step 612, using the formula Δf∝TBW, where total body water is proportional to the change in resonance. At step 614, fat mass is estimated using the equation m_{fat}=m_{body}−m_{water}. wherein m_{water} is mass of total body water. The ΔQ is the change in Q factor observed in the resonant cavity and is an important measure to reduce the effects of sample shape on the overall TBW measurement. Comprehensive body composition, mass, and height measurements are taken at step 316. A risk assessment of developing metabolic disease is determined at step 320, based on the measurements.

In embodiments, the present specification describes devices and methods for measuring body composition to provide a quantifiable measure of individual metabolic health in adults and pediatrics. In addition, pediatric descriptors of health using body composition may be provided.

In embodiments, the methods of the present specification derive predictive models for measuring body composition in arbitrarily shaped phantoms over a wide range of masses to provide an understanding of how shape may affect body mass measurements; using the predictive models, eliminate shape as a factor, by making adjustments to the basic resonant properties of the resonant cavity; and use mass and RCP to accurately measure body composition.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive.

We claim:

1. A system for generating a body composition measurement of a biological body, comprising:
a resonant cavity comprising a frame and metal material lining the frame, wherein the resonant cavity has a top panel, bottom panel, and four side panels to thereby define an enclosed volume;
at least one antenna in the resonant cavity;
a network analyzer in data communication with the at least one antenna; and
a controller configured to receive data from the network analyzer and generate an output, wherein the output comprises data indicative of the body composition of the biological body.

2. The system of claim 1 further comprising a scale in physical communication with the resonant cavity and configured to measure a weight of the biological body in the enclosed volume, wherein the controller is further configured to receive data from the scale and generate an output, wherein the output comprises data indicative of the weight of the biological body.

3. The system of claim 1 further comprising a height measurement system coupled to the resonant cavity and configured to measure a height of the biological body in the enclosed volume, wherein the controller is further configured to receive data from the height measurement system and generate an output, wherein the output comprises data indicative of the height of the biological body.

4. The system of claim 1 further comprising an optical shape measurement system coupled to the resonant cavity and configured to measure a shape of the biological body in the enclosed volume, wherein the controller is further configured to receive data from the optical shape measurement system and generate an output, wherein the output comprises data indicative of the shape of the biological body.

5. The system of claim 1 further comprising an oscillator in data communication with the network analyzer.

6. The system of claim 1 wherein at least one of the four side panels is a door and is configured to open and close in order to provide access to the enclosed volume.

7. The system of claim 1 further comprising a second antenna, wherein the at least one antenna and the second antenna are turned to two different orthogonal directions in the resonant cavity.

8. The system of claim 7 further comprising a third antenna, wherein the at least one antenna, the second antenna, and the third antenna are each turned to different orthogonal directions in the resonant cavity.

9. The system of claim 1, wherein the controller is configured to generate the body composition measurement of biological body by measuring a cavity resonance and a cavity Q factor of the resonant cavity when empty, measuring a mass of the biological body, measuring a body resonance and a body Q factor of the biological body, calculating differences or shifts of the cavity resonance relative to the body resonance and the cavity Q factor relative to the body Q factor, adjusting for fill factor of the cavity, calculating a total body water, and estimating a fat mass of the body.

10. The system of claim 9, wherein the controller is further configured to determine a risk assessment of developing metabolic disease based on the generated body composition measurement.

11. A method for generating a body composition measurement of a biological body, comprising:
providing a body composition measurement system comprising:
a resonant cavity comprising a frame and metal material lining the frame, wherein the resonant cavity has a top panel, bottom panel, and four side panels to thereby define an enclosed volume;
at least one antenna in the resonant cavity;
a network analyzer in data communication with the at least one antenna; and
a controller configured to receive data from the network analyzer and generate an output;
measuring a cavity resonance and a cavity Q factor of the cavity when empty;
measuring a mass of the biological body placed within the cavity;
measuring a body resonance and a body Q factor of the biological body placed within the cavity;
determining a differential between the measured resonances and Q factors;
measuring a total water content of the biological body by using the determined differential; and
generating data indicative of the body composition of the biological body by using the measured mass and total water content of the biological body.

12. The method of claim 11, wherein the system further comprises a scale in physical communication with the resonant cavity and configured to measure a weight of the biological body in the enclosed volume, the method further comprising receiving data from the scale and generating an output, wherein the output comprises data indicative of the weight of the biological body.

13. The method of claim 11, wherein the system further comprises a height measurement system coupled to the resonant cavity and configured to measure a height of the biological body in the enclosed volume, wherein the method further comprises receiving data from the height measurement system and generating an output, wherein the output comprises data indicative of the height of the biological body.

14. The method of claim 11, wherein the system further comprises an optical shape measurement system coupled to the resonant cavity and configured to measure a shape of the biological body in the enclosed volume, wherein the method further comprises receiving data from the optical shape measurement system and generating an output, wherein the output comprises data indicative of the shape of the biological body.

15. The method of claim 11, wherein the system further comprises an oscillator in data communication with the network analyzer.

16. The method of claim 11, wherein at least one of the four side panels is a door and is configured to open and close in order to provide access to the enclosed volume.

17. The method of claim 11, wherein the system further comprises a second antenna, wherein the at least one antenna and the second antenna are turned to two different orthogonal directions in the resonant cavity.

18. The method of claim 17, wherein the system further comprises a third antenna, wherein the at least one antenna, the second antenna, and the third antenna are each turned to different orthogonal directions in the resonant cavity.

19. The method of claim 11, wherein the controller is configured to generate the body composition measurement of biological body by calculating differences or shifts of the cavity resonance relative to the body resonance and the cavity Q factor relative to the body Q factor, adjusting for fill factor of the cavity, calculating a total body water, and estimating a fat mass of the body.

20. The method of claim 19, wherein the network analyzer is further configured to determine a risk assessment of developing metabolic disease based on the generated body composition measurement.
